(19) United States
(12) Patent Application Publication
(10) Pub. No.: US 2019/0046734 A1
(43) Pub. Date: Feb. 14, 2019

(54) TORSION SPRING FIXATION IN AUTOMATIC DRUG DELIVERY DEVICE

(71) Applicant: Novo Nordisk A/S, Bagsvaerd (DK)

(72) Inventors: Nikolaj Eusebius Jakobsen, Soeborg (DK); Christian Peter Enggaard, Vejby (DK)

(21) Appl. No.: 15/763,491

(22) PCT Filed: Sep. 29, 2016
(86) PCT No.: PCT/EP2016/073330

§ 371 Date: Mar. 27, 2018

(30) Foreign Application Priority Data
Oct. 1, 2015 (EP) 15187857.6

(51) Int. Cl. A61M 5/315; A61M 5/20

(52) U.S. Cl. CPC A61M 5/31593; A61M 5/2033; A61M 5/31553; A61M 2005/3154; A61M 5/31536; A61M 5/31583; A61M 5/31541

(57) ABSTRACT

A drug injection device (1) comprising a dose expelling mechanism for expelling drug from a reservoir through a reservoir outlet, the dose expelling mechanism comprising: a first component (40), a second component (10, 10′, 15′, 10″, 15″), and a torsion spring element (20) for inducing relative rotation between the first component (40) and the second component (10, 10′, 15′, 10″, 15″) to execute a dose expelling operation, the torsion spring element (20) extending along a longitudinal axis and comprising: a first spring end portion (22) being rotationally restrained with respect to the first component (40), and a second spring end portion (23) exhibiting an end portion inner diameter in a relaxed state of the torsion spring element (20), wherein the second spring end portion (23) is fitted over a spring receiving portion (11) of the second component (10, 10′, 15′, 10″, 15″), the spring receiving portion (11) having a transversal dimension which is larger than the end portion inner diameter, and the fitting of the second spring end portion (23) over the spring receiving portion (11) being configured to provide a sticking friction engagement between the two when the torsion spring element (20) is strained to exert a maximum in-use torque.

### FIELD OF THE INVENTION

The present invention relates to torsion spring powered drug delivery devices.

### BACKGROUND OF THE INVENTION

Automatic pen-type injection devices where a user strains a torsion spring during dose setting and where the torque stored in the torsion spring is subsequently utilised to expel a liquid drug from a reservoir have been known for decades and are at present e.g. used to administer insulin or glp-1 in the treatment of diabetes mellitus. U.S. Pat. No. 5,104,380 (Owen Mumford Limited) provides early examples of such automatic injection devices.

The torsion spring used in an automatic injection device is typically a helically coiled spring. The helical spring is operationally positioned between the device housing and a rotatable dose setting member and is strained by rotation of the latter relative to the former. Conventionally, hooks are formed at the respective spring ends and used to anchor the spring to e.g. the aforementioned components.

WO 2006/045526 (Novo Nordisk A/S) provides an example of a helical torsion spring having a radially outwardly protruding portion at the one end for attachment to a housing of an injection device and a radially inwardly protruding portion at the other end for attachment to a rotatable dose setting member. Further, WO 2012/063061 (Owen Mumford Limited) discloses various examples of hooked spring ends being secured to different parts of an injection device.

While attachment by hooked spring ends may be a technically satisfactory solution providing hooks in the first place is associated with additional costs. Hence, it is more expensive to produce helical torsion springs with hooked ends than helical torsion springs with straight ends.

In addition thereto, hooked spring ends have very poor rotational tolerance in the sense that it is difficult to ensure during production of the spring that the hooks are arranged exactly such that they both later on during assembly angularly align with a receiving lug in the injection device when the spring is in a completely relaxed state. A torsion spring is typically prestrained during assembly of the injection device to provide for an ever present minimum torque level which is sufficient to overcome the friction in the dosing mechanism and ensure complete delivery of a set dose, regardless of its size. In order to minimise a variation of the drug delivery profile over a batch of injection devices it is desirable to achieve a reproducible mounting and a predictable pre-straining of the individual springs.

It is thus desirable to provide a torsion spring based automatic injection device employing a spring type which is cheaper to produce and which entails an easier assembly process and yields an even more predictably operating end product.

### SUMMARY OF THE INVENTION

It is an object of the invention to eliminate or reduce at least one drawback of the prior art, or to provide a useful alternative to prior art solutions.

In particular, it is an object of the invention to enable the provision of a torsion spring based automatic drug delivery device employing a spring component which is cheaper to produce than the conventionally used springs.

It is a further object of the invention to enable the provision of a torsion spring based automatic drug delivery device which can be easily assembled in a manner which yields consistent results.

It is an even further object of the invention to enable the provision of a torsion spring based automatic drug delivery device which has a well-defined drug expelling profile.

In the disclosure of the present invention, aspects and embodiments will be described which will address one or more of the above objects and/or which will address objects apparent from the following text.

A drug delivery device, such as e.g. a drug injection device, embodying the principles of the invention has a dose expelling mechanism for expelling drug from a reservoir through a reservoir outlet, and the dose expelling mechanism comprises a first component, a second component, and a torsion spring element for inducing relative rotation between the first component and the second component to execute a dose expelling operation and thereby cause a dose of drug to be expelled from the reservoir. The torsion spring element extends along a longitudinal axis and comprises a first spring end portion which is rotationally restrained with respect to the first component, and a second spring end portion which exhibits an end portion inner diameter in a relaxed state of the torsion spring element and which is fitted over a spring receiving portion of the second component. The spring receiving portion has a transversal dimension which is larger than the end portion inner diameter, and the fitting of the second spring end portion over the spring receiving portion is configured to provide a sticking friction engagement between the two when the torsion spring element is strained to exert a maximum in-use torque.

Accordingly, in one aspect of the invention, a drug delivery device may be provided comprising a dose expelling mechanism which includes a first component, a second component, an actuation element, and a torsion spring element. The torsion spring element extends along a longitudinal axis and is capable of inducing relative rotation between the first component and the second component to move the actuation element. The torsion spring element comprises a first spring end portion which is rotationally restrained with respect to the first component, and a second spring end portion. The second spring end portion exhibits an end portion inner diameter in a relaxed state of the torsion spring element, i.e. in an unloaded equilibrium state of the torsion spring element prior to installation in the drug delivery device. The second spring end portion is fitted over a spring receiving portion of the second component, the spring receiving portion having a transversal dimension which is larger than the end portion inner diameter. Further, the fitting of the second spring end portion over the spring receiving portion is configured to provide a sticking friction engagement between the second spring end portion and the spring receiving portion when the torsion spring element is strained to exert a maximum in-use torque.

Thereby, a fully functioning automatic drug delivery device is enabled based on a torsion spring element where at least one of the spring ends can be of straight configuration, i.e. not hooked. This reduces the cost of producing the torsion spring element and allows for an easier and more reliable mounting of the torsion spring element during assembly of the drug delivery device, as the spring end portion can be fitted over the spring receiving portion in any angular orientation of the spring end portion relative to the longitudinal axis. It is thus not required that two separate hooked spring ends have a particular relative angular orientation for the torsion spring element to engage properly with respective receiving portions in the drug delivery device.

The drug delivery device may be of the type having an irreplaceably attached reservoir or of the type comprising means for receiving a user attachable reservoir.

In the present context when a spring end portion is “rotationally restrained” with respect to a component at least a portion of the particular spring end portion is prevented from angular displacement relative to the particular component in at least one of the two possible directions of rotation about the longitudinal axis, i.e. at least a portion of the spring end portion is rotationally fixed in at least one rotational direction with respect to the component. As an example, a spring end portion terminating in an open hook will engage more tightly with a stationary component when rotated in one direction and disengage from the stationary component when rotated in the opposite direction, whereas a spring end portion terminating in a tightly closed hook will engage more tightly with the stationary component when rotated in either direction.

Further, in the present context, the term “maximum in-use torque” refers to the maximum torque which the torsion spring element will be able to exert during use of the drug delivery device. In the course of various user actions pertaining to a normal use of the drug delivery device the torsion spring element may be twisted various degrees. The “maximum in-use torque” reflects the torque exerted by the torsion spring element when exhibiting the maximum possible degree of twisting during use of the drug delivery device. The maximum possible degree of twisting may be correlated with the maximum dose expellable in one dose expelling operation.

The sticking friction engagement between the second spring end portion and the spring receiving portion guarantees that the torsion spring element does not slip over the surface of the second component when strained to exert a torque between the first component and the second component. In case the torsion spring element is a helical spring to obtain a sticking friction engagement which is reliable during all stages of normal use of the drug delivery device the various components may be chosen and dimensioned such that

$$EID \frac{{R}_{2} - {R}_{1}}{2 {R}_{1} {R}_{2}^{2}} ( {e}^{2 N πμ} - 1 ) \geq {T}_{ma x}$$

where E is the elastic modulus of the torsion spring element, I is the area moment of inertia of the torsion spring element, D is the diameter of the spring receiving portion, R₁ is the radius of the neutral axis of the second spring end portion when in a free, unexpanded state, R₂ is the radius of the neutral axis of the second spring end portion when wrapped around the spring receiving portion, N is the number of spring coils being wrapped around the spring receiving portion, μ is the coefficient of friction, and Tₘₐₓ is the maximum in-use torque of the torsion spring element. If the drug delivery device e.g. offers expelling of the drug in doses of different sizes, satisfying the above expression will ensure a sticking friction engagement between the second spring end portion and the spring receiving portion even when the largest dose is selected.

In particular embodiments of the invention the drug delivery device is an injection device, such as e.g. a pen-type injector. Pen-type injectors are attractive due to their slender configuration and accordingly widely used, e.g. in the treatment of diabetes mellitus. Such devices typically employ a cartridge type drug reservoir characterised by having a hollow reservoir body, e.g. generally cylindrical with a constriction at one end, for accommodating the drug, which reservoir body is sealed by a penetrable septum (e.g. self-sealing), respectively a slidable piston.

In a drug delivery device employing a cartridge type reservoir the actuation element may be a piston rod which extends along a piston rod axis and which is adapted to move along the piston rod axis during dose expelling to thereby pressurise the reservoir by applying a force to the piston.

The drug delivery device may further comprise a housing for accommodating the dose expelling mechanism, or at least a portion thereof. In a practically simple embodiment of the invention one of the first component and the second component is arranged stationarily relative to the housing and the other of the first component and the second component is rotatable relative to the housing about the longitudinal axis.

The drug delivery device may also further comprise a dose release structure, such as e.g. a dose release button, operable to activate the dose expelling mechanism and thereby cause a dose to be expelled.

Hence, specifically a drug delivery device may be provided comprising a housing and a dose expelling mechanism for expelling drug from a reservoir through a reservoir outlet, the housing accommodating at least a portion of the dose expelling mechanism, and the dose expelling mechanism comprising A) a first component, B) a second component, C) a torsion spring element for inducing a relative rotational movement between the first component and the second component by release of stored energy, the torsion spring element extending along a longitudinal axis and comprising c1) a first spring end portion being rotationally restrained with respect to the first component, and c2) a second spring end portion exhibiting an end portion inner diameter in a relaxed state of the torsion spring element, D) a piston rod extending along a piston rod axis, the piston rod being operatively coupled with the housing, and with one of the first component and the second component during the relative rotational movement between the first component and the second component, and being configured to move along the piston rod axis in response to the relative rotational movement between the first component and the second component, and E) a dose release structure operable to cause the torsion spring element to release stored energy, wherein the second spring end portion is fitted over a spring receiving portion of the second component, the spring receiving portion having a transversal dimension which is larger than the end portion inner diameter, and the fitting of the second spring end portion over the spring receiving portion being configured to provide a sticking friction engagement between the two when the torsion spring element is strained to exert a maximum in-use torque.

The piston rod may be operatively coupled with the housing via engagement with a nut member, the nut member either forming part of the housing or being a separate element rotationally fixed to the housing. Alternatively, the piston rod may be operatively coupled with the housing via a longitudinally extending spline connection.

The drug delivery device may further comprise a dose setting mechanism allowing a user to select a dose among a plurality of doses for expelling by the dose expelling mechanism, e.g. as conventionally known in the art of injection devices. The dose setting mechanism may comprise a user operable dose setting button being rotatable about the longitudinal axis to set the dose to be expelled. The dose setting button may be rotatable between a zero dose set position in which no dose is selected, and a maximum dose set position in which the maximum settable dose has been selected.

The second component may be axially movable relative to the housing between a first position in which the second component and the dose setting button are rotationally interlocked and a second position in which the second component and the dose setting button are rotationally decoupled. Thereby, when the second component is in the first position and the dose setting button is rotated to set a dose the spring receiving portion is angularly displaced, and the torsion spring element accordingly strained. A ratchet coupling between the dose setting button and the housing, which may be overcome by the user providing a dose setting torque, may be provided to prevent the torsion spring element from being prematurely released when the user lets go of the dose setting button during or after dose setting. Hence, when the second component is in the first position the drug delivery device is in a dose setting state.

The dose setting mechanism may further allow the user to adjust a set dose by either increasing or decreasing the set dose as well as to cancel a set dose by resetting.

The second component may be operatively coupled with the dose release button and configured to move from the first position to the second position in response to the dose release button moving relative to the housing from a dose ready position to a dose release position. During movement to the second position the second component may slide into rotational interlocking engagement with a piston rod guide, which piston rod guide is rotatably mounted in the housing and in rotational interlocking connection with the piston rod. The piston rod may be rotatably mounted in a guide structure in or of the housing, e.g. by threaded engagement with an internal nut member, such that a torque applied to the piston rod results in a helical displacement of the piston rod along the longitudinal axis. In that case, when the second component is in the second position the torsion spring element is released and the drug delivery device is in a dose expelling state.

The piston rod guide may be capable of unidirectional rotation only relative to the housing to thereby prevent a rotation that would lead to an undesired proximal displacement of the piston rod, away from the reservoir.

In some embodiments of the invention the dose release button is arranged at a proximal end of the housing and configured to be moved axially between a proximal position corresponding to the dose ready position and a distal position corresponding to the dose release position, e.g. by the user applying a depressive force by use of a thumb or a forefinger.

The dose release button may be biased towards the proximal position, e.g. by a compression spring, a foam pad, or the like, such that when the depressive force from the user is interrupted the dose release button is automatically returned to the proximal position.

The second component may be further configured to move from the second position to the first position in response to the dose release button moving from the dose release position to the dose ready position. Thereby, when the dose release button is returned to the dose ready position the second component is automatically moved into rotational interlocking connection with the dose setting button and the drug delivery device is switched to the dose setting state.

In some embodiments of the invention the second component comprises a first part and a second part arranged end to end and connected in a friction fit or snap fit connection. An end portion of the first part thereby surrounds an end portion of the second part in an overlap zone. At least a portion of the overlap zone is surrounded by the spring receiving portion and the torsion spring element thus exerts a compressional force to the overlap zone when mounted over the spring receiving portion, thereby strengthening the friction fit or snap fit connection between the first part and the second part. This makes the two-part second component less flexible and the joint more torque resistant.

The first spring end portion may exhibit a second end portion inner diameter in the relaxed state of the torsion spring element. This second end portion inner diameter may be equal to, smaller than, or greater than the end portion inner diameter of the second spring end portion. The first spring end portion may be fitted over a spring receiving portion of the first component having a transversal dimension which is larger than the second end portion inner diameter, and the fitting of the first spring end portion over the spring receiving portion of the first component may be configured to provide a sticking friction engagement between the two when the torsion spring element is strained to exert the maximum in-use torque.

Thereby, the torsion spring element may be realised with two straight ends, lowering the production costs even further. Also, the torsion spring element may be attached to the first component and the second component during assembly of the drug delivery device by very simple respective expansions over dedicated portions of the two components.

The torsion spring element may be or comprise a helical spring, a torsion rod, a torsion tube, or the like. For example, the torsion spring element may be a helical spring having a constant inner diameter along its entire length, from a first spring end to a second spring end, when in the relaxed state.

In particular embodiments of the invention the torsion spring element is a helical spring having a plurality of windings, and the second spring end portion comprises at least one winding and at most five windings. This means that at least one winding and at most five windings are fitted over the spring receiving portion, having due consideration to the above mathematical expression. Thereby, as little of the length of the torsion spring element as possible will be allocated to the coupling with the second component, minimising the number of inactive windings that are useless vis-à-vis the energy storage capacity of the torsion spring element.

Similarly, the first spring end portion may comprise at least one winding and at most five windings.

In another aspect of the invention, an injection device is provided comprising a housing and a dose expelling mechanism for expelling a dose of drug from a drug reservoir, wherein the dose expelling mechanism comprises a piston rod, a rotatable piston rod drive structure being rotationally interlocked with the piston rod during dose expelling, and a helical spring structure adapted to provide energy for rotation of the rotatable piston rod drive structure. The piston rod is configured for translational or helical displacement through a guide portion in the housing in response to a relative rotation between the rotatable piston rod drive structure and the housing. The helical spring structure comprises a first spring end portion being rotationally restrained with respect to the housing, a second spring end portion being rotationally restrained with respect to the rotatable piston rod drive structure, and a coil body extending between the first end portion and the second end portion. One of the first spring end portion and the second spring end portion has an end portion inner diameter when in a relaxed state and is wrapped around a geometry having a transversal dimension which is larger than the end portion inner diameter.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 shows a torsion spring as used in state of the art automatic injection devices,

FIG. 2 shows a drive member and a torsion spring member of a drive assembly for use in a drug delivery device according to an embodiment of the invention,

FIG. 3 is a longitudinal perspective section view of the drive assembly members of FIG. 2,

FIG. 4 is a perspective cut-out of a drive member in an alternative configuration, and

FIG. 5 is a longitudinal section view of a proximal portion of a drug delivery device according to another embodiment of the invention.

In the figures like structures are mainly identified by like reference numerals.

### DESCRIPTION OF EXEMPLARY EMBODIMENTS

When in the following relative expressions, such as “upper” and “lower”, are used, these refer to the appended figures and not necessarily to an actual situation of use. The shown figures are schematic representations for which reason the configuration of the different structures as well as their relative dimensions are intended to serve illustrative purposes only.

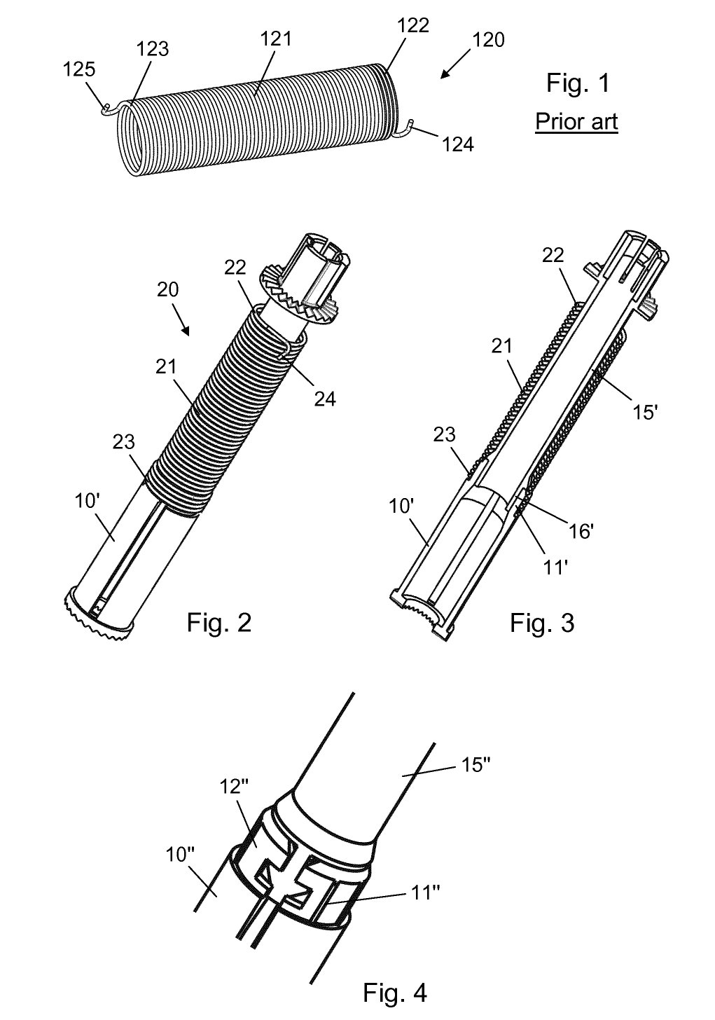

FIG. 1 shows a torsion spring 120 as conventionally used in the art of torsion spring based automatic injection devices. The torsion spring 120 comprises a helically coiled body 121 which extends longitudinally between a proximal end portion 122 and a distal end portion 123. The proximal end portion 122 terminates in a proximal (open) hook 124 while the distal end portion 123 terminates in a distal (open) hook 125. In conventional applications the proximal hook 124 is attached to a proximal receiving section which is stationary with respect to the injection device housing, and the distal hook 125 is attached to a distal receiving section arranged on a rotatable drive member capable of causing activation of a piston rod by rotation about its own axis of extension. The torsion spring 120 thus serves as a rotational energy storage component and is arranged to exert a torque to rotate the drive member during release of the stored energy.

In relation to the manufacturing of such a torsion spring the bending of a spring end is a cost increasing procedure. Furthermore, given the intended use of the torsion spring in a high precision drug injection device the position of the hooked ends relative to one another is of great importance, since the manufacturer optimally wants each hooked end to engage the dedicated receiving section in the injection device in a fully relaxed state of the torsion spring, such that a predictable torque level is produced in the torsion spring during the subsequent pre-straining. Such pre-straining is performed in order to ensure that a certain torque level is always available in the torsion spring so that even the smallest dose can be expelled. In other words it is important for this type of torsion spring that the hooked ends are arranged such that they align with their respective receiving sections during assembly of the injection device. This is a difficult and even further cost increasing task.

According to the present invention, this task can be eliminated by using an arrangement as indicated in FIGS. 2, 3 and 5.

FIGS. 2 and 3 show, in a perspective view, respectively a longitudinal perspective section view, a longitudinally extending torsion spring 20 arranged about a two-component drive member 10′, 15′ consisting of an actuation member 10′ and a transmission tube 15′ arranged in longitudinal extension of one another and operatively coupled by a snap fit connection.

The function of the drive member 10′, 15′ is similar to that of an alternative single-component drive tube 10 described in detail below in the context of an injection device. Thus, the two-component embodiment simply serves to illustrate the principle solution to the above highlighted challenges.

The torsion spring 20 comprises a helical coil body 21, a proximal end portion 22 terminating in a hook 24, and a distal end portion 23, which is wrapped around a spring receiving portion 11′ of the actuation member 10′. It is noted that in a relaxed state, i.e. before attachment to the actuation member 10′, the torsion spring 20 has a constant spring inner diameter, d. However, since the spring receiving portion 11′ has an outer diameter, D, which is greater than the spring inner diameter, d, the distal end portion 23 is actually radially expanded over the spring receiving portion 11′, and as a result a sticking friction engagement between the two is provided due to the compressional force exerted by the distal end portion 23.

The number of windings and the radial expansion of the distal end portion 23 about the spring receiving portion 11′ required to hold a specific torque exerted by the torsion spring 20 can be determined from the belt friction equation. The spring/drive member connection should be dimensioned to hold a specific torque which corresponds to the maximum in-use torque of the torsion spring 20, i.e. the torque exerted by the torsion spring 20 when strained to deliver the maximum dose offered by the injection device. Hence, the following expression must be satisfied:

$$EID \frac{{R}_{2} - {R}_{1}}{2 {R}_{1} {R}_{2}^{2}} ( {e}^{2 N πμ} - 1 ) \geq {T}_{ma x}$$

where E is the elastic modulus of the spring, I is the area moment of inertia of the spring, D is the diameter of the clutch arbor (the spring receiving portion), R₁ is the radius of the neutral axis of the spring end portion when in a free, unexpanded state, R₂ is the radius of the neutral axis of the spring end portion when wrapped around the spring receiving portion, N is the number of spring coils being wrapped around the spring receiving portion, μ is the coefficient of friction, and Tₘₐₓ is the maximum in-use torque of the spring.

The fact that the distal end portion 23 is wrapped around the spring receiving portion 11′ in this manner and thereby is rotationally fixed with respect to the actuation member 10′ eliminates the need for a hook at this end of the torsion spring 20. The cost of producing the torsion spring 20 is thereby reduced. Furthermore, the assembly process is simplified because only one hooked spring end needs to be aligned with a dedicated receiving section. The distal end portion 23 will be in sticking friction engagement with the spring receiving portion 11′ regardless of the angular orientation of the distal end portion 23.

Notably, the wrapped distal end portion 23 additionally tightens the snap fit connection between the actuation member 10′ and the transmission tube 15′ in that the compressional force exerted on the spring receiving portion 11′ tends to press the spring receiving portion 11′ radially against a coupling portion 16′ of the transmission tube 15′. The tightened snap fit connection increases the rotational stiffness of the two-component drive member 10′, 15′ and improves its reliability as a torque resisting structure.

FIG. 4 shows a portion of an alternative two-component drive member 10″, 15″ consisting of an actuation member 10″ and a transmission tube 15″ arranged in longitudinal extension of one another and operatively coupled by a snap fit connection, similar to the snap fit connection of the drive member 10′, 15′ shown in FIGS. 2 and 3.

The actuation member 10″ has a proximal interface 12″ which is adapted for interaction with the torsion spring 20. The proximal interface 12″ has a circular-cylindrical basic shape of diameter, d, but is provided with a plurality of circumferentially distributed, longitudinally extending ridges 11″, whereby the transversal dimension of the proximal interface 12″ is increased so that a radial expansion of the distal end portion 23 is needed for it to fit over the proximal interface 12″. An arrangement is thus provided whereby the distal end portion 23 may be rotationally fixed to a spring receiving section in a sticking friction engagement, similarly to what is described above, without requiring the basic shape diameter of the spring receiving section be greater than the spring inner diameter, d.

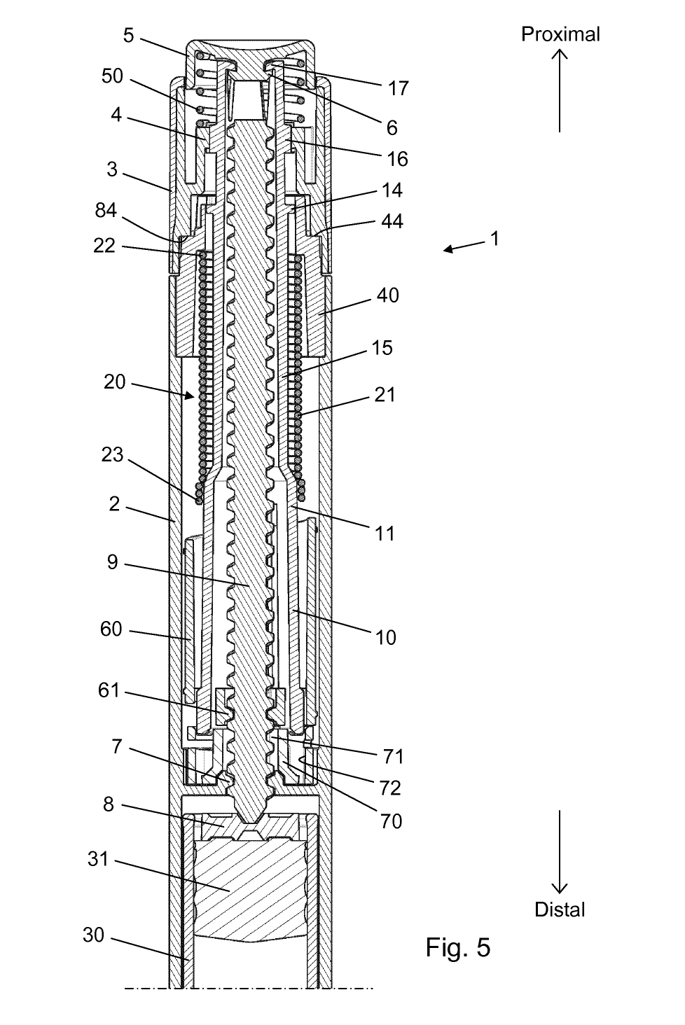

FIG. 5 shows an exemplary use of the torsion spring 20 in a pen-type drug injection device 1. Only a proximal portion of the injection device 1 is shown. The undisclosed distal portion may be realised as such are conventionally in the art of pen-type injection devices.

The injection device 1 has a generally circular-cylindrical housing 2 which accommodates a portion of a drug containing cartridge 30, being sealed by, respectively, a slidable piston 31 and a needle-penetrable rubber septum (not shown), as well as a drive mechanism for advancing the piston 31 through the cartridge 30. A rotatable, 2K moulded, dose dial 3 is arranged at the proximal end of the housing 2 for allowing a user to select a dose to be delivered from the cartridge 30.

A piston rod 9 extends longitudinally within the housing 2 and is in threaded engagement with a nut member 7 thereof. The piston rod 9 has a distal end face which during use of the injection device 1 abuts a piston washer 8 serving as a force distributor for the piston 31. The piston rod 9 further has a longitudinally extending groove (not visible) providing for rotational interlocking engagement with a piston rod guide 70, a key 71 of the latter being slidably received in the groove.

The piston rod guide 70 is axially fixed in, but capable of unidirectional rotation relative to, the housing 2. On an interior surface the piston rod guide 70 is provided with teeth 72 adapted for disengageable engagement with mating teeth 19 on an exterior portion of a drive tube 10.

The drive tube 10 is a unitary structure which extends axially in the housing 2 and is arranged about a portion of the piston rod 9. A distal portion of the drive tube 10 has longitudinal slits (not visible) to allow an otherwise exteriorly arranged scale drum 60 to extend radially through the drive tube 10 and threadedly engage with the piston rod 9 via an interior nut member 61. In addition to coupling the scale drum 60 directly to the piston rod 9 this provides a rotational interlocking connection between the drive tube 10 and the scale drum 60.

A proximal portion 15 of the drive tube 10 comprises a circumferential toothing 18 adapted for disengageable engagement with an interior toothed collar 4 of the dose dial 3, and a radially inwardly extending catch portion 17 firmly gripping a harpoon member 6 of a proximal injection button 5, thereby providing a translational interlocking connection between the drive tube 10 and the injection button 5. The injection button 5 is axially movable between an inactive position (shown in FIG. 5), in which the injection device 1 is in a dose setting state, and an active position, in which the injection device 1 is in a dose delivery state. A compression spring 50 arranged to act between an interior surface of the injection button 5 and an upper transversal surface of the toothed collar 4 biases the injection button 5 towards the inactive position. The proximal most position of the injection button 5 relative to the housing 2 is defined by a flange 14 provided on the proximal portion 15 of the drive tube 10, which flange 14 serves to limit proximal movement of the drive tube 10 relative to a spring base 40 being axially and rotationally fixed with respect to the housing 2.

The drive mechanism is powered by the torsion spring 20. The hook 24 of the proximal end portion 22 is rotationally anchored to the spring base 40, in a manner conventionally known in the art, and the spring base 40 thus provides a stationary reference point for the torsion spring 20. The distal end portion 23 is wrapped around a spring receiving portion 11 of the drive tube 10 so as to establish a rotational interlocking connection between the distal end portion 23 and the drive tube 10. The spring receiving portion 11 has an outer diameter, D, which is larger than the inner diameter, d, of the torsion spring 20, which means that the distal end portion 23 is elastically expanded over the spring receiving portion 11 and thus exerts a compressional force thereonto. In the shown embodiment three windings of the torsion spring 20 overlap the spring receiving portion 11 and thus ensure a sticking friction engagement with the drive tube 10.

The spring base 40 has a proximally facing serrated surface 44 capable of slipping engagement with a distally facing serrated interior surface 84 of the dose dial 3, thereby providing a ratchet mechanism for preventing relaxation of the torsion spring 20 during dose setting.

In the following a situation of use of the injection device 1 will be described.

The injection device 1 is prefilled in the sense that it carries the cartridge 30 when delivered from the manufacturer, and the basic user steps required to administer a dose of the contained drug (e.g. insulin, glp-1 or a mixture thereof) are simple and relatively fast to execute: 1) attach an injection needle assembly (not shown) to the distal end of the cartridge 30, 2) dial a desired dose, 3) insert the injection needle at a suitable injection site, and 4) initiate an expelling of the set dose by releasing the torsion spring 20. Steps 1) and 3) may be performed in accordance with common ways of attaching an injection needle assembly to a pen-type injection device and subsequently inserting the injection needle into the skin, and since these steps are irrelevant to the description of the present invention they will not be discussed any further in this text.

So, in order to set a dose to be administered the user may hold the housing 2 in one hand and use the other hand to rotate the dose dial 3 relative to the housing 2, about a longitudinal axis of the injection device 1. The injection button 5 is in its inactive position, i.e. its proximal most position relative to the housing 2, due to the biasing force from the compression spring 50. Due to the engagement between the harpoon member 6 and the catch portion 17 the drive tube 10 is accordingly situated in a proximal most position. In the proximal most position of the drive tube 10 the circumferential toothing 18 is radially aligned with the interior toothed collar 4, thereby rotationally interlocking the drive tube 10 and the dose dial 3. A rotation of the dose dial 3 about the longitudinal axis, in either direction, thus results in a corresponding rotation of the drive tube 10.

The rotation of the drive tube 10 occasions both a helical travel of the scale drum 60 within the housing 2 and a twisting of the torsion spring 20. Due to the rotational interlocking connection between the drive tube 10 and the scale drum 60 the scale drum 60 is forced to rotate in accordance with the rotation of the drive tube 10, and as the piston rod 9 is held stationary by the piston rod guide 70, the threaded engagement between the piston rod 9 and the nut member 61 causes the scale drum 60 to move helically about the piston rod 9. The scale drum 60 carries a plurality of dose related indicia (not shown), and a window (not visible) in the housing 2 allows the user to see a subset of these dose related indicia pass by as the scale drum 60 moves and to determine the size of the set dose from the current position of the scale drum 60. Furthermore, as the spring receiving portion 11 of the drive tube 10 is rotated relative to the spring base 40 the wrapped distal spring end portion 23 is angularly displaced relative to the proximal spring end portion 22, an amount corresponding to the angular displacement of the spring receiving portion 11 due to the sticking friction engagement between the two, whereby rotational energy is stored in the torsion spring 20. The ratchet connection between the proximally facing serrated surface 44 of the spring base 40 and the distally facing serrated interior surface 84 of the dose dial 3 allows the user to set the desired dose by rotating the dose dial 3 relative to the housing 2 in discrete steps, and to reduce a set dose by rotating the dose dial 3 in the reverse direction.

In order to expel a set dose from the cartridge 30 the injection button 5 is depressed, whereby the compression spring 50 is compressed and the drive tube 10 is moved distally in the housing 2. The distal movement of the drive tube 10 causes the circumferential toothing 18 to slide out of engagement with the interior toothed collar 4 and the teeth 19 to slide into engagement with the mating teeth 72 on the interior surface of the piston rod guide 70. Notably, the resulting rotational interlocking of the drive tube 10 with the piston rod guide 70 takes effect before complete disengagement of the circumferential toothing 18 from the interior toothed collar 4.

At some point during the depression of the injection button 5 the circumferential toothing 18 disengages completely from the interior toothed collar 4, whereby the energy stored in the torsion spring 20 is released and the distal end portion 23 is returned to its pre-dose setting position. The rotation of the distal end portion 23 causes a corresponding rotation of the drive tube 10 due to the sticking friction engagement between the wrapped spring windings and the spring receiving portion 11.

Due to the established rotational interlocking engagement between the drive tube 10 and the piston rod guide 70 the piston rod guide 70 is forced to rotate, whereby the key 71, being engaged in the longitudinally extending groove of the piston rod 9, causes a corresponding rotation of the piston rod 9, which is then helically advanced through the nut member 7 to displace the piston washer 8 and the piston 31 distally in the cartridge 30.

Simultaneously, the scale drum 60 rotates along with the drive tube 10, and is thereby helically displaced in the distal direction along with the piston rod 9, until a portion of the scale drum 60 meets a rotational stop surface (not visible) in the housing 2. At this stop surface the scale drum 60 is in an end-of-dose position in the housing 2.

As the rotation of the scale drum 60 stops, so does the rotation of the drive tube 10 and thereby also the rotation of the piston rod guide 70 and the piston rod 9 is resultantly halted in the nut member 7. When the user discontinues her force on the injection button 5 the compression spring 50 expands and returns the injection button 5 to the inactive position. The drive tube 10 is thereby moved proximally in the housing 2 until the flange 14 meets a stop surface (not visible) on the spring base 40. In this position of the drive tube 10 the circumferential toothing 18 has re-engaged with the interior toothed collar 4 and the teeth 19 have disengaged from the teeth 72, rotationally decoupling the drive tube 10 and the piston rod guide 70. The injection device 1 is now ready for the setting of a new dose.

It is noted that even though the injection device 1 exemplifies the invention by the distal end portion 23 of the torsion spring 20 being wrapped around the spring receiving portion 11, an alternative embodiment could be envisaged in which the proximal end portion 22 was wrapped around e.g. an axially protruding structure in the spring base 40 and the distal end portion 23 had a hook for attachment to the drive tube 10. In a further alternative embodiment both the proximal end portion 22 and the distal end portion 23 could be wrapped around suitable geometries on, respectively, the spring base 40 and the drive tube 10.

1. A drug injection device comprising a dose expelling mechanism for expelling drug from a reservoir through a reservoir outlet, the dose expelling mechanism comprising:
a first component,
a second component, and
a torsion spring element for inducing relative rotation between the first component and the second component to execute a dose expelling operation, the torsion spring element extending along a longitudinal axis and comprising:
a first spring end portion being rotationally restrained with respect to the first component, and
a second spring end portion exhibiting an end portion inner diameter in a relaxed state of the torsion spring element,
wherein the second spring end portion is fitted over a spring receiving portion of the second component, the spring receiving portion having a transversal dimension which is larger than the end portion inner diameter, and the fitting of the second spring end portion over the spring receiving portion being configured to provide a sticking friction engagement between the two when the torsion spring element is strained to exert a maximum in-use torque.

2. A drug injection device according to claim 1, further comprising a housing accommodating at least a portion of the dose expelling mechanism,
wherein one of the first component and the second component is arranged stationarily relative to the housing, and the other of the first component and the second component is capable of rotation relative to the housing about the longitudinal axis.

3. A drug injection device according to claim 2, further comprising a dose setting mechanism for setting of a dose of drug to be expelled by the dose expelling mechanism, the dose setting mechanism comprising a dose setting button being rotatable about the longitudinal axis between a zero dose set position and a maximum dose set position by a user providing a torque to overcome a ratchet coupling between the dose setting button and the housing,
wherein the second component is axially movable between a first position in which the spring receiving portion is rotationally interlocked with the dose setting button and a second position in which the spring receiving portion is rotationally decoupled from the dose setting button.

4. A drug injection device according to claim 3, further comprising a dose release button for activating the dose expelling mechanism to expel a set dose of drug,
wherein the second component is adapted to, during movement from the first position to the second position, rotationally interlock with a rotatable piston rod guide being rotationally interlocked with the piston rod, and
wherein the second component is operatively coupled with the dose release button and configured to move from the first position to the second position in response to the dose release button moving relative to the housing from a proximal position to a distal position.

5. A drug injection device according to claim 4, wherein the second component is further configured to move from the second position to the first position in response to the dose release button moving relative to the housing from the distal position to the proximal position.

6. A drug injection device according to claim 5, wherein the dose release button is biased towards the proximal position.

7. A drug injection device according to claim 1, wherein the second component comprises a first part and a second part arranged end to end and friction fitted in an overlap zone, wherein at least a portion of the overlap zone is surrounded by the spring receiving portion.

8. A drug injection device according to claim 1, wherein the first spring end portion exhibits a second end portion inner diameter in the relaxed state of the torsion spring element, and
wherein the first spring end portion is fitted over a spring receiving portion of the first component, the spring receiving portion of the first component having a transversal dimension which is larger than the second end portion inner diameter, and the fitting of the first spring end portion over the spring receiving portion of the first component being configured to provide a sticking friction engagement between the two when the torsion spring element is strained to exert the maximum in-use torque.

9. A drug injection device according to claim 1, wherein the torsion spring element is a helical spring, and
wherein the second spring end portion comprises at least one winding and at most five windings.

10. A drug injection device according to claim 9, wherein the first spring end portion comprises at least one winding and at most five windings.

11. A drug injection device according to claim 9, wherein the helical spring has a constant inner diameter when in the relaxed state.
